(19) United States
(12) Patent Application Publication
(10) Pub. No.: US 2014/0187437 A1
(43) Pub. Date: Jul. 3, 2014

(54) Cross-Reactive Sensor Arrays And Methods of Use

(71) Applicant: The Trustees Of Columbia University In The City Of New York, New York, NY (US)

(72) Inventors: Milan Stojanovic, Fort Lee, NJ (US); Tilla Worgal, New York, NY (US); Kyungae Yang, New York, NY (US)

(73) Assignee: The Trustees Of Columbia University In The City Of New York, New York, NY (US)

(21) Appl. No.: 14/040,242

(22) Filed: Sep. 27, 2013

Related U.S. Application Data
(63) Continuation of application No. PCT/US2012/031563, filed on Mar. 30, 2012.
(60) Provisional application No. 61/469,789, filed on Mar. 30, 2011.

(51) Int. Cl. G01N 33/68

(52) U.S. Cl. CPC G01N 33/6803
USPC 506/9; 506/16

(57) ABSTRACT

Disclosed herein are methods and compositions directed to the construction of aptamer-based receptors and cross-reactive sensor arrays of such receptors and the use of the same in disease detection and management.

### CROSS-REFERENCE TO RELATED APPLICATIONS

This application is a continuation of PCT Application No. PCT/US12/31563, filed Mar. 30, 2012, which claims the benefit of and priority to U.S. Provisional Application Ser. No. 61/469,798, filed Mar. 30, 2011, both of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Jan. 7, 2014, is named 070050-5054_SL.txt and is 5,881 bytes in size.

### BACKGROUND OF THE INVENTION

Cross-reactive sensor arrays of receptors (chemical/electronic noses) are inspired by the abilities of the mammalian olfactory system to differentiate a large number of odorants over a wide range of concentrations and against various backgrounds. In mammalian olfaction the sparseness of the receptor space is overcome by a complex computing process in the neural system.

Non-specific hydrophobic receptors suitable for the construction of cross-reactive sensor arrays have been previously disclosed. In more recent work, methods for the construction of high-resolution cross-reactive sensor arrays having a minimal number of individual receptors are described. For example, a cross-reactive sensor array comprising three receptors capable of identifying ten hydrophobic analytes over a range of concentrations has been constructed.

However, one drawback of these earlier approaches is that a large number of individually synthesized receptors containing modified bases have to be screened in order to identify optimal subsets of receptors for any one particular cross-reactive sensor array. Accordingly, there remains a need in the art for techniques of rapidly identifying such individual receptors and the resulting cross-reactive sensor arrays as well as for applying such cross-reactive sensor arrays to medical problems. The present invention addresses these needs.

### SUMMARY OF THE INVENTION

In certain embodiments, the present invention is directed to methods of synthesizing and training aptamer-based receptors using SELEX and/or an equivalent directed evolution technology.

In certain embodiments the present invention is directed to cross-reactive sensor array compositions comprising one or more of aptamer-based receptors. In certain embodiments, the cross-reactive sensor array comprises an aptamer-based receptor capable of binding an analyte in a manner indicative of a disease state and an aptamer-based receptor capable of binding an analyte in a manner indicative of the absence of the disease state.

In certain embodiments, the present invention is directed to methods of detection a disease state by comparing contacting a cross-reactive sensor array with a patient sample, where the pattern of individual receptors bound by analytes present in the sample are indicative of a disease state. In certain embodiments, the present invention is directed to methods of detection a disease state by comparing contacting a cross-reactive sensor array with a patient sample, where the pattern of individual receptors bound by analytes present in the sample are indicative of the absence of the disease state.

### BRIEF DESCRIPTIONS OF THE DRAWINGS

FIG. 1A-B. (A). Fully matched three-way junction with an analyte (black circle); bold lines represent additional bases (SEQ ID NO: 6). (B). A randomized library of hydrophobic pockets based on three way junctions with N representing any of the four bases (A, T, C, G) (SEQ ID NO: 7).

FIG. 2A-B. (A). Selection design: A library N8 is attached to a column via a “competitor” oligonucleotide; those members that are specifically washed away from the columns by addition of steroids are amplified and used in the next round of selection (SEQ ID NOS. 8, 9, 8 and 9, respectively, in order from left to right). (B). Structures of the four steroids used in selections.

FIGS. 3A-3D depict four aptamers used in a cross-reactive array and the results achieved with each. FIG. 3A discloses SEQ ID NO: 10 on the left and SEQ ID NO: 11 on the right. FIG. 3B discloses SEQ ID NO: 12 on the left and SEQ ID NO: 13 on the right. FIG. 3C discloses SEQ ID NO: 12 on the left and SEQ ID NO: 14 on the right. FIG. 3D discloses SEQ ID NO: 15 on the left and SEQ ID NO: 16 on the right.

FIG. 4 depicts a 3D-presentation-sensor based steroid classification.

FIG. 5A-D. The selection procedure is represented here schematically (using hypergraphs), with an example of expected results. (A) A junction space (based on the collection described herein) is represented as an n-gon (with 48 vertices in the first selection). A training selection with positive samples in the [DISEASE] class is presented as a selection of a common hyper-edge (simplex) connecting the most abundant responsive junction vertices (above 10% participation in the pool after all cycles of selection; actual numbers are hypothetical)—in this case a hyper-edge actually has a physical analog in the MB oligonucleotide, while numbers are also ‘weights’ indicating the ratio of responsiveness of junctions. (B) The same procedure is repeated with the negative [HEALTH] class, providing five different most abundant surviving junctions in this pool. (C) Two subsets are combined (union A&B) to yield a cross-reactive array predicted to have very high classification power (e.g., 99%), most likely even with linear classifier or simple decision trees. This array is minimized in (D) using individual members of a training sample set, with a minimal loss in classification accuracy (e.g., >98%).

FIG. 6 depicts Four alkaloids (1, 2, 3 and 6) separated over a range of concentrations by two sensor junctions, B and D. These can be classified with a simple decision tree with clearly defined cut-off ranges.

FIG. 7 depicts a cross-reactive array SELEX scheme.

FIG. 8A-C. (A) Steroid and oligonucleotide (herein with a stem-loop structure and a fluorophore) compete for a junction (S with quencher), which leads to a release of oligonucleotide in a way that depends on the concentration of steroids. The beacon stem-loop structure gives us greater variability in signaling; and in this case a c(S)>c(MB) set up is used. The beacon can also be labeled with both fluorophore and quencher, in which case free beacon will be of low fluorescence. FIG. 8A discloses SEQ ID NOS 17, 18, 17 and 18, respectively, from left to right. (B) The principle of junction culling (or enrichment) that is used to perform training (SELEX): junctions that bind steroids in the sample will pass through the column (survive), because steroids compete out oligonucleotide deposited on a column. (C) This design could be used to select junctions from a library that strongly bind to steroids: starting with a collection of randomized junctions (N is any of the nucleotides, A, T, C, or G; this particular collection (library) has 8 randomized positions, total of 48 unique structures). The sample is added, and passed over the column displaying the beacon. Pass-through consists of those junctions that bind to steroids. Less tight binding can be allowed for by continuing to elute the column with the sample. Primers are for the PCR amplification. FIG. 8C discloses SEQ ID NOS 19 and 18, respectively, from left to right.

FIG. 9A-C. (A) A blown-up region of a microchip (Combimatrix) with twelve electrodes and dimensions, with three proposed structures of junctions different in one position (red) next to three electrodes. One helix, which interacts with both competitor oligonucleotide (at 5′ end) and through which TWJ is attached to the surface (at 3′ end) has fixed structure (boxed), leading to 8 randomized positions in the initial calculations below. Of note, individual TWJs are separated from one another by single point mutations. (B) Assay with a molecular beacon (F—fluorophore, e.g., Cy5) in solution interacting with a junction on the surface of the chip. In the presence of steroid, the beacon is captured less efficiently, leading to a drop in fluorescence proportional to the strength of junction-steroid interactions. The lengths of the individual oligonucleotides can be optimized to achieve the best signal (x, y, z, v are lengths of individual regions). (C) Alternative assay that uses the same format is depicted.

FIG. 10 depicts a hypothetical example including a 3D presentation of results separated into three classes and where G, D, and B axis are ratios of changes in the intensity of individual sensor dyes to standard dye.

FIG. 11 depicts the corticosteroids in urine. Free cortisol represents 1% of urinary steroids.

### DETAILED DESCRIPTION OF THE INVENTION

In certain embodiments, the present invention is directed to methods and compositions that enable SELEX/microarray screening directly on patients' samples to isolate classifying cross-reactive arrays. The production of such classifying cross-reactive arrays is unprecedented and, with appropriate procedural modifications, as outlined herein, it is generally useful in a variety of medical contexts.

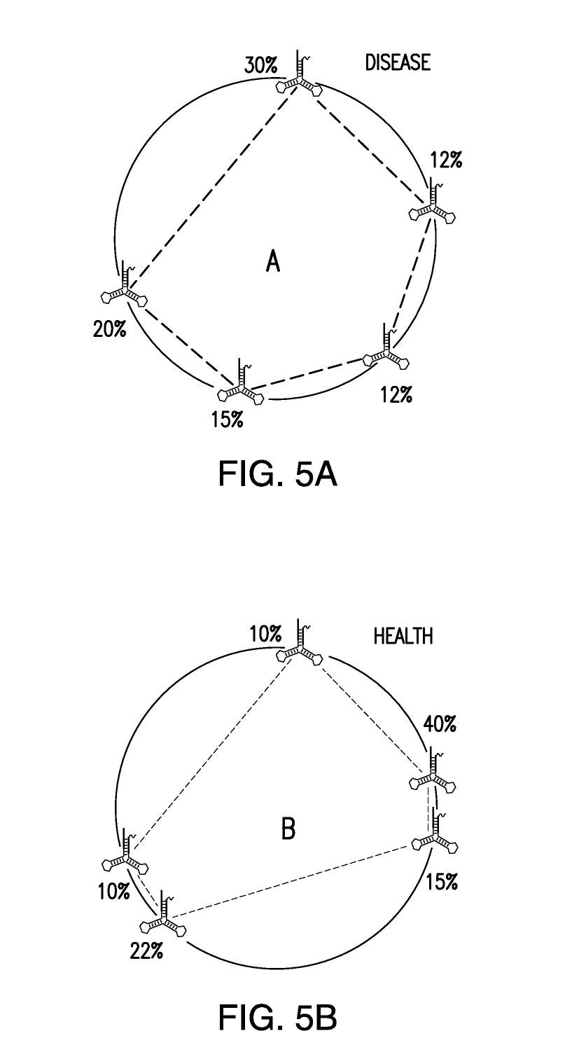

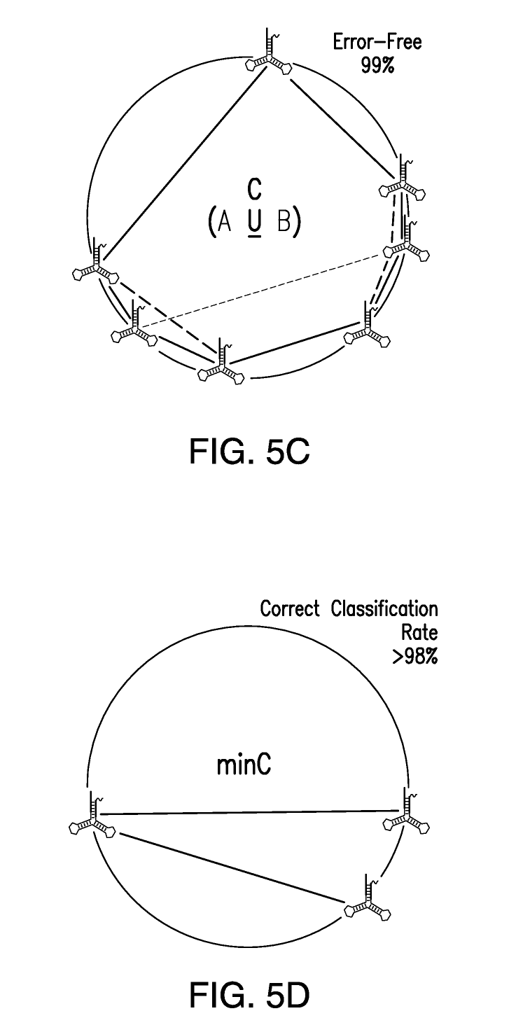

In certain embodiments, the present invention is directed to identifying: (i) a first subset of receptors interacting with what is common in samples displaying the characteristic molecular pattern of some disease (FIG. 5A); and (ii) a second subset of receptors interacting with what is common in healthy samples (FIG. 5B). These subsets can be combined (FIG. 5C) into a classifying cross-reactive sensor array, which can then be minimized (FIG. 5D). In certain embodiments, the procedure is repeated on multiple classes of samples and, if analytes are similar, common minimizations can be performed. In certain embodiments, the result will be a general diagnostic classifier (e.g., for multiple diseases, indicating both disease presence and type) taking the form of a cross-reactive sensor array.

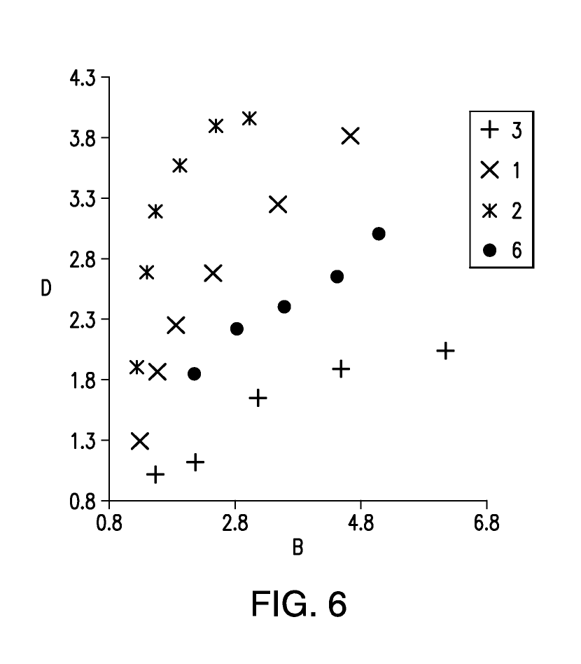

The concept underlying certain embodiments of the present invention is retrospectively straightforward and is based on the ability to cover the receptor space efficiently and in a focused manner. This involves a search (and minimization) for pairs of sensors (e.g., B and D) that will be differentially reactive for two classes, i.e., at all concentrations one sensor will be more responsive to one class then other and vice versa. This leads to two classes being separated easily by a simple decision tree method (if B<D then [DISEASE]) or even by a simple linear classifier. Using this approach it is possible in certain embodiments, with substantial coverage of receptor space and a suitable search method, to identify two sensors that will allow a simple decision tree-based classification of samples into a number of classes (such as the four classes identified in FIG. 6). For example, it was known in the art that two sensors could be used to classify a pair of analytes, and that up to ten analytes over a full range of concentrations could be classified with three optimal sensors with linear classifiers and decision trees. FIG. 6 depicts an example of four alkaloids being classified by two sensors, over a range of concentrations.

In certain embodiments of the present invention, non-specific receptors, i.e. patter-based sensing rather than analyte-specific sensing, are employed in the context of the cross-reactive sensor array rather than specific analyte receptors. Embodiments employing such non-specific receptors are useful in situations where: (i) non-pattern-based sensing requires standardization of results against creatinine; (ii) specific receptors would be limited to single analytes, and would fail with variations within families of analytes (which is often the case with metabolic diseases); (iii) three specific receptors would suffice to classify ten analytes as they may in the example, thus, the arrays may lead to faster indication of the possible diagnosis; and (iv) non-specific receptors would be able to indicate that there is a gross change in the pattern even for a disease that is not otherwise precisely characterized. The last scenario is in fact common with rare inborn errors, where even an indication of disease presence can lead to low-cost generic therapies.

In certain embodiments, the methods of the present invention differ from traditional approaches to cross-reactive sensor arrays that try to closely mimic the mammalian olfactory system. In contrast to these conventional methods, the compositions and methods of such embodiments of the present invention enjoy the advantage of being able to expand receptor space (essentially) endlessly, so the computational complexity of training and classification can be replaced with the discovery of the best receptors for specific tasks under natural conditions. This realization has another consequence: the receptors of certain embodiments of the present invention are synthetic compounds and can be reproducibly made anywhere in the world. This circumvents a traditional limitation of cross-reactive arrays: the need for retraining.

In certain embodiments, the junctions of the present invention are non-responsive to non-steroid small-molecule components of urine (e.g., uric acid, hippuric acid, creatinine, and amino acids) providing an excellent opportunity to focus on the rapid and general detection of cumulative gross shifts in composition of steroids regardless of their exact structure. In contrast to standard spectrophotometric methods, the compositions and methods of such embodiments of the instant invention are not expected to be impacted by interference from other small molecules that have no large hydrophobic surfaces or that have radically different shapes.

In certain embodiments, the compositions and methods of the present invention have the ability to improve current diagnostic approaches in large number of clinically relevant conditions, extend diagnostic capacity to low-prevalence conditions that remain undiagnosed due to economical and technical reasons, and, to an extent, uncover “unrecognized” alterations in metabolism. In certain embodiments, the approach of the instant invention is particularly powerful when a health problem is characterized through a shift in patterns of families of metabolites or surface markers, rather than a single distinguishing component. In certain cases, the individual component analysis will give only a partial answer and differential diagnosis requires multiple steps. In certain embodiments, the instant invention can replace these multiple steps with a single test indicative of the final diagnosis, allowing early focus of the clinicians' effort.

In certain embodiments, the compositions and methods of the instant invention can be used in connection with the detection of inborn errors and diseases of steroid metabolism detectable through urinalysis. Urine is one of the most readily accessible samples from humans. In 1862 a leading English physician of his time, William Roberts, wrote that “the amount of information concerning a urine which may be obtained through the unaided senses of smell and sight far exceeds, both in precision and extent, what is usually supposed . . . ”. This ability has been almost completely lost in modern clinicians, due to the focus on specific tests. The cross-reactive arrays outlined herein can significantly change the current practice, allowing additional valuable information to be mined from urines by a simple procedure.

Human urine contains an abundance of various steroids and their conjugates; the composition and relative ratios of steroids are critical indicators of the physiological status of the organism. The intractable nature of this mixture and the variations in matrix urine properties and the kidneys' filtration efficacy lead to tests that often complicated and rarely cost-effective. The standard and proposed methods to determine specific steroids include various immunoassays, while mixtures are analyzed by LC-MS, and MS (including tandem) methods, or various enzyme-coupled assays; these are time-consuming and almost always require pretreatment (e.g., deconjugation and/or solid phase extraction). If the methods are antibody-based, they are usually specific for a single component, necessitating a large number of antibodies to cover a large number of diseases. These problems are compounded by the fact that samples need to be standardized against creatinine levels. Finally, various metabolic errors (e.g., liver failure) might not even be characterized by a specific single component, but rather by a family. The exact members of the family that are increased may be patient specific (due to differences in metabolism, gender, drugs inhibiting metabolism, etc.).

In certain embodiments, the compositions and methods of the present invention can be used in the analysis of “free cortisol” samples. This determination is currently used for the initial screening that, after multiple additional tests, leads to a differential diagnosis of at least four different diseases. The sensitivity of the sensors described herein is well within the reference values for secondary tests such as 17 KS (17-ketosteroids, range from 200-800 μM in positive samples) and 17 OHCS (17-hydroxy corticosteroids, 50-270 μM in positive samples) in 24-h urines, used to assess further adrenal abnormalities. These samples are thus the first choice for selection and the immediate diagnostic advantage over the standard clinical practice is demonstrated through their use, for example, the fact these samples will not have to be separately collected.

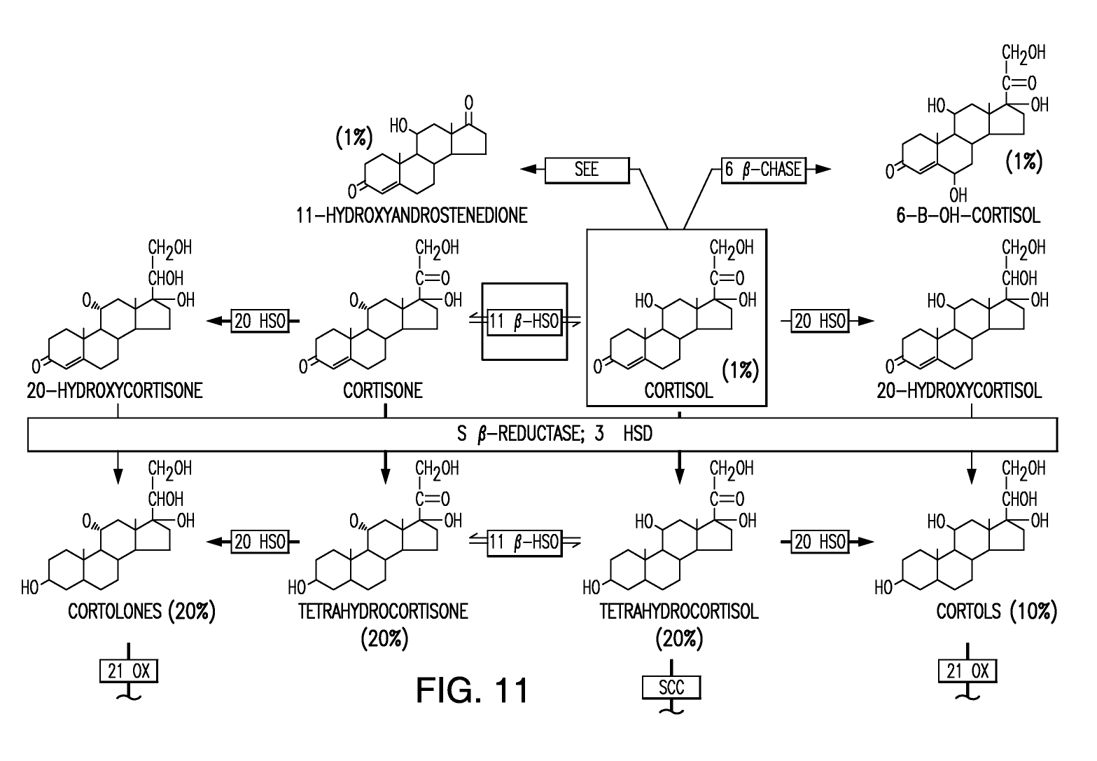

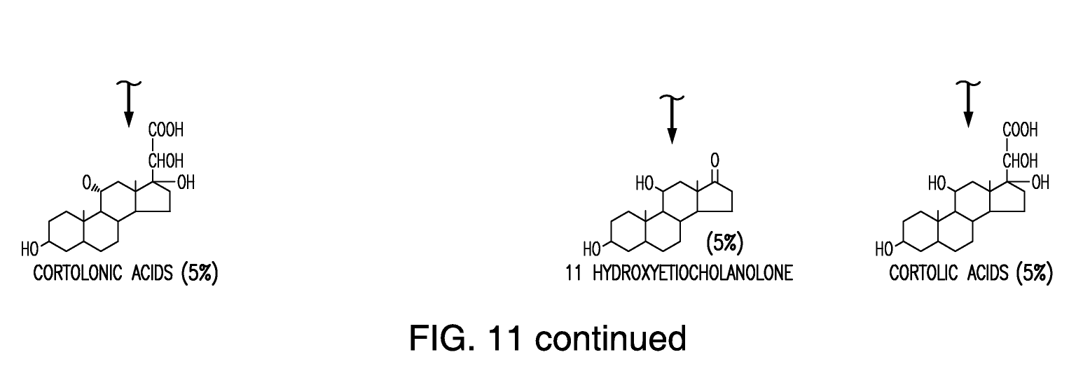

Current practice is to determine free cortisol (UFC) (28-124 nmol/L) The free cortisol test, an integrated measure of plasma free cortisol, measures 1% of excreted corticosteroids (FIG. 11). UFC replaced determination of the significantly more abundant 17-hydroxycorticosteroids (<28 umol/24 h) or 17-ketosteroids (<63 umol/24 h), due to less dependency on accurate urine collection. Determination of UFC is a good screening test but necessitates further determination for final diagnosis. It fails in up to 15% of Cushing's patients; is not a good measure for adequacy of steroid supplementation does not cover the rare, yet significant congenital adrenal hyperplasias and requires normalization to creatinine and a 24 h urine collection. Conversely, moderately elevated results always need be endorsed by further testing before making a diagnosis of Cushing's syndrome. The technology disclosed herein, in contrast, allows a comprehensive evaluation in random urine

Another example of an application where certain embodiments of the present invention can have immediate impact is routine monitoring of liver function in patients at risk for developing liver failure (e.g., during drug trials, parenteral nutrition, or liver transplants). Healthy adults excrete moderate quantities of bile acids into urine (˜6.6 μmol/24 h) but patients with cholestasis can excrete 100 μmol and more within 24 h. This test, in an array format, could become standard and widely used bedside/at-home test. For example, in certain embodiments, the test will comprise an array with an electrical readout, similar to current glucose monitoring, being provided to all patients at risk, with sudden shifts in bile acids signaling the need for follow up with specific change in treatment.

Bile acids are produced in the liver, secreted in response to meals and undergo efficient enterohepatic circulation. Bile acids are the major catabolic pathway of cholesterol, necessary for lipid and vitamin absorption. Urinary bile acids (UBA), mainly conjugated with sulfate followed by glucuronic acid and amino acids, provide an integrated index of bile acid metabolism with the advantage that they are not affected by meals (in contrast to serum bile acids).

UBA are sensitive markers for liver diseases. Liver diseases can be roughly divided to be of heaptocellular, cholestatic or mixed origin. The diagnostic spectrum of UBA is not limited to cholestatic disease but has been shown to include diseases of hepatocellular origin, i.e. of viral and alcoholic origin in which inflammation and necrosis predominate, as well as inborn errors of bile acid metabolism (7 primary defects, all rare; 3 secondary defects: Zellweger's Syndrome, Smith-Lemli Opitz Syndrome) that account for 2% of unexplained liver disease in infants and children. In healthy individuals, urinary bile acid excretion is negligible. Cholestasis secretion of increases urinary bile acid excretion that can be readily detected and are proportional to the degree of cholestasis and such changes can be detected using the compositions and methods of certain embodiments of the instant invention.

Important and largely unmet areas of a need for rapid, cost effective, solution-phase tests in medical diagnostics are the assessment of patterns of steroid excretion in urine in the context of the expansion of a comprehensive program for mass screening for inborn errors of metabolism and continuous monitoring of infants with diseases. The increases in concentrations of dominant components in metabolic errors are truly gross. In congenital adrenal hyperplasia (CAH) the increase in concentrations of the most characteristic single steroid is from 80 μM (healthy) to 300-1300 μM (CAH samples), the latter depending on the exact forms of CAH. In another example, the life-saving treatment with bile acids is considered to be of the utmost importance in several inborn errors of bile acid metabolism or chronic cholestatic conditions in newborns; these cause on average a 100-fold increase in total bile acid concentrations (from av. 1.5 μM in healthy to av. 150 μM in afflicted infants) in urine. While there is a mandatory screening for a single form of CAH, screening for other diseases is not considered cost-effective, due to a low incidence and individual variabilities of dominant components (ruling out antibody-based methods). In certain embodiments, the compositions and methods of the present invention will allow for the bundled detection of rare disease with CAH, and indicate the need for further screening and lifesaving therapy. The method could be cost-effective for initial testing in the developing world, where MS-based screening is unavailable, as well as in the developed world for the monitoring of treatment.

In certain embodiments, the compositions and methods of the present invention can be used to identify oligosaccharides markers. Complex oligosaccharide markers can be used in neonatal screening, but this problem is even more intractable than in the case of steroid metabolism. In certain embodiments, the receptors of the present invention can be focused on a “sugar component” and that can be accomplished by using boronic acids derivatives in the procedures described herein, preselecting against simple mono- and disaccharides. In addition, in certain embodiments, PCR-amplifiable bases, with different polymerases (e.g., Vent), can be used or microarray-based selections can be used.

In order to improve differential response of sensors to dominant components in individual classes, and achieve better classification, a counter-selection protocol can be introduced in certain embodiments. In certain embodiments, this would include washing, in early elutions, with a counter-selection agent, e.g., a steroid that would be typical for dominant component for another sample. For example, but not by way of limitation, if aptamers for [Increased Corticosteroids] class are being selected for, a prewash with one bile acid (e.g., deoxycholic acid) could be used as the counter-selection agent.

In certain embodiments collections of aptamer-based receptors can be employed that are overlapping in binding capacity. Thus, in certain embodiments, the analytes bound by said aptamer-based receptors will form overlapping populations.

As outlined above and in the Examples that follow, certain embodiments of the TWJ/SELEX-based technology of the present invention can achieve at least equal quantitative and qualitative evaluation of urinary excretion patterns of adrenal steroids and bile acids as conventional techniques at a significantly lower cost. In addition, in certain embodiments, this technology is suitable for point-of-care that does not necessitate a 24 h urine collection or expensive and physically large machinery. Thus, certain embodiments of the present technology provide more information, at a lower cost and a fast turn-around time, than a determination of free cortisol. For example, adrenal neoplasia would reveal a quantitative increase in corticosteroids, while 11-β-HSD deficiency dependent hypertension would reveal a change in cortisol/cortisone ratio. The current LCMS approach is labor intensive and dependent on expensive equipment. The instant technology merely necessitates a cocktail of defined receptors, i.e. a cross-reactive sensor array, which is added to a random urine sample and read in a standard fluorometer. One estimate is that the basic costs for the test would be less than $1 per sample.

### EXAMPLES

### Example 1

### 1.1. Selection Design

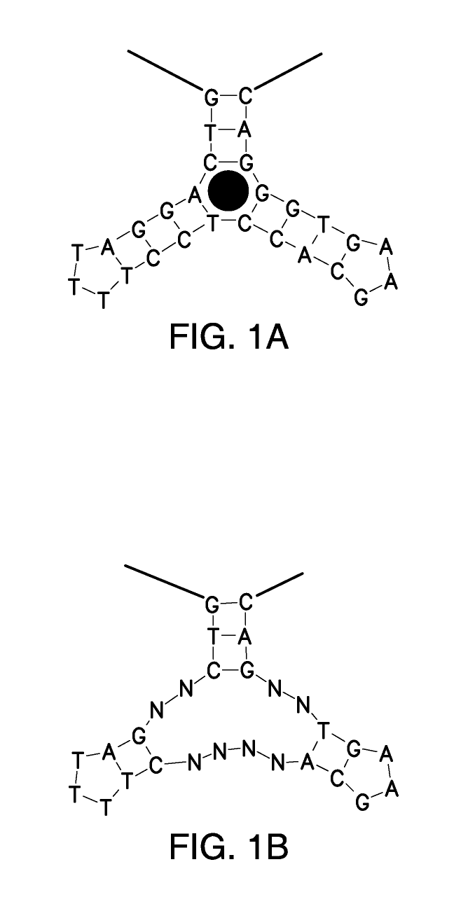

Setting up a selection requires defining the initial library of three-way junctions and selection conditions for interactions between this library and steroid samples. The procedure was tested using a highly focused library of three way junctions with eight randomized positions (FIG. 1b), i.e., with 48 members. This library is significantly less rich than those used conventionally employed in SELEX procedures, which typically sample from the 4⁴⁰ receptor space (i.e., with a 40-mer randomized region). However, for proof of concept purposes and to allow for evaluation of evolutionary processes, a library that can be always reproduced in next selections, and even on microarrays, if necessary (to allow for in silico evolution), a library in which differences in results could not be attributed to random distributions of sequences was considered suitable. However, as detailed below, because any synthetic library can include errors introduced during synthesis (despite initial stringent gel purification) and/or PCR amplification, additional sequence diversity.

All oligonucleotides were obtained from Integrated DNA Technologies (Coralville, Iowa, USA). The oligonucleotides for a library and primers were used without further purification, for the modified oligonucleotide types (e.g. biotinylation, fluorophore conjugation, etc), HPLC-purified grade oligonucleotides were used. All compounds were purchased from Sigma-Aldrich Co. (St. Louis, Mo., USA) unless otherwise noted. DEPC-treated and nuclease-free water (Fisher Scientific; Fair Lawn, N.J., USA) was used for all purpose, e.g. oligonucleotide dissolution and buffer preparation.

Preparation of buffer and steroid solution. Three different buffers were prepared; 1) SELEX reaction buffer (20 mM Tris, pH 7.4, 1 M NaCl, 10 mM MgCl2); 2) No MgCl washing buffer (20 mM Tris, pH 7.4, 1 M NaCl), and 3) 2× SELEX reaction buffer (40 mM Tris, pH 7.4, 2 M NaCl, 20 mM MgCl2).

The target molecules are 4 different steroids; deoxycholic acid (DCA), dehydroisoandro sterone-3-sulfate (DIS), deoxycorticosterone-21 glucoside (DOG) and β-estradiol. The 100 mM of steroid stock solutions for DCA, DIS, DOG and 50 mM for β-estradiol were prepared by the manufacture's recommendation and then the stock was diluted half-in-half by SELEX reaction buffer. The serial dilution range is from 375 uM to 6 uM.

In vitro selection process. The oligonucleotides for steroids SELEX are as follow: (1) Random (N8) library (78 mer): 5′-GGTATTGAGGAGGCTCTCG-GGACGAC(N2)GGATITTTCC(N4)ACGAAGT(N2)GTCGTCCCGATCCTCCTAACGTACGA CT-3′ (SEQ ID NO: 1), (2) Forward-primer (24 mer): 5′-GGTATTGAGGAGG-CTCTCGGGACG-3′ (SEQ ID NO: 2), (3) Reverse-primer (20 mer): 5′-AGTCGTACGTTAGGAGGATC-3′ (SEQ ID NO: 3), (4) Biotinyl-ated reverse-primer (20 mer): 5′-biotin-AGTCGTAC-GTTAGGAGGATC-3′ (SEQ ID NO: 4), (5) Biotinylated column immobilizing sequence (I-DNA; 25 mer): 5′-GGTCC-GTCGTCCCGAGAGCCGGACC-biotin-3′ (SEQ ID NO: 5).

For the 1st round of SELEX, the oligonucleotide mixture containing 0.25 nmole I-DNA and 0.05 nmole random library was prepared by 250 ul of SELEX reaction buffer and incubated at 95° C. for 5 min. The mixture samples were cooled down to room temperature (>10 min), then added to a streptavidin agarose column. A streptavidin agarose column was prepared with 250 ul of the streptavidin agarose resin (1-3 mg Biotinylated BSA/ml resin; Theromo scientific, Ill., USA) in a micro bio-spin chromatography column (Bio-rad, CA, USA). For equilibrium, streptavidin agarose was washed 5 times with the same volume of SELEX reaction buffer, and then the oligonucleotide mixture was flowed through the column. The mixture was collected and applied to column again. After washing 10 times, the DNA-steroid complex is eluted with 250 ul of 20 uM steroid solution, repeated 2 times. The eluted samples were used as template for PCR after concentration up to 50 ul by Amicon Ultra centrifugal filter (Milipore, Cork, Ireland).

PCR condition following protocol: 1 cycle of 95° C., 2 min, 13 cycle of [95° C., 15 sec; 57.5° C., 30 sec; 72° C., 45 sec], and 1 cycle of 72° C., 2 min. The PCR amplicons were concentrated by the centrifugal filter, and the original library size of product was analyzed using 4% E-gel (Invitrogen, CA, US) and purified with GIAEX II gel purification kit (QIAGEN, Maryland, USA).

Following purification, the double strand PCR amplicons were captured on streptavidin agarose column via the biotinylated antisense strand; 0.2 ml streptavidin agarose resins were filled and washed 3 times by the strand separation buffer (20 mM Tris, pH 7.4, 1 M NaCl). After loading the sample into the column, the resin was washed 5 times with the strand separation buffer, and incubated with 0.20 ml of 0.2M NaOH. The flow-out drops were collected, neutralized and concentrated using a centrifugal filter for the next round of SELEX as a library.

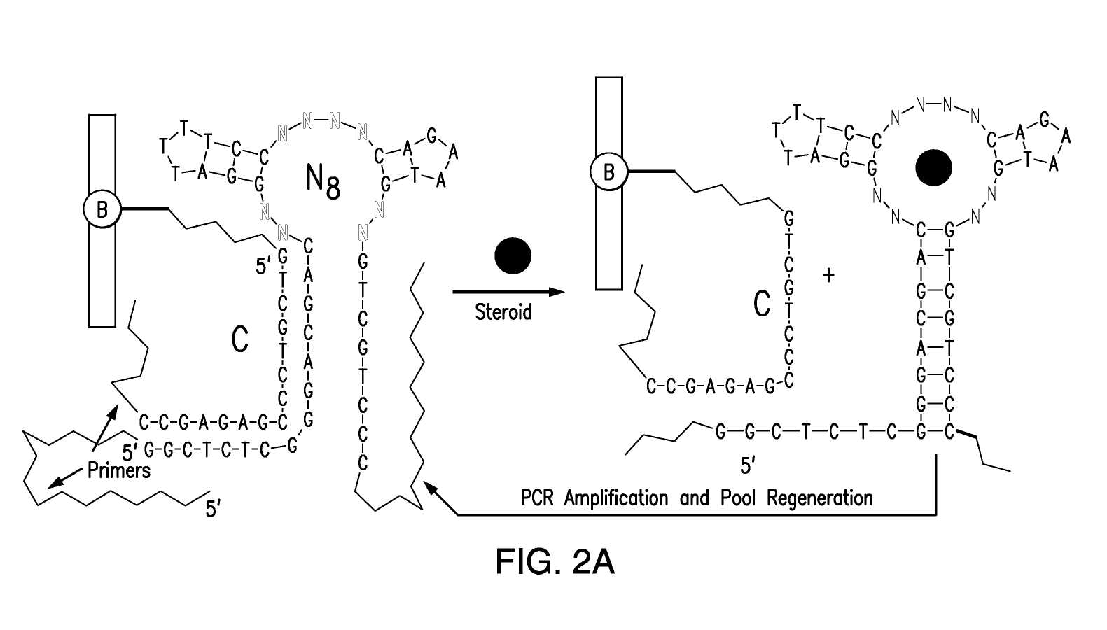

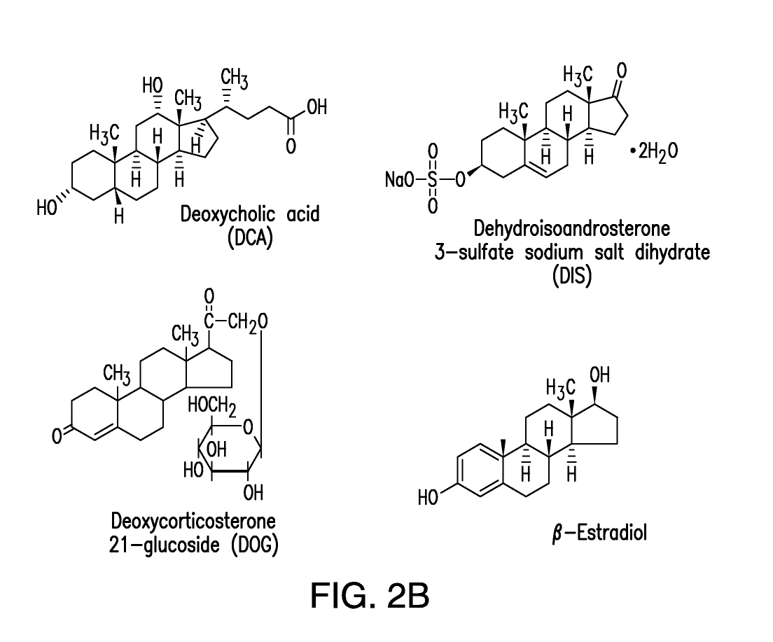

Because next selections will be done on urine samples, a procedure in which affinity material is solid cannot be used. There is a single report of SELEX using solution phase non-oligonucleotide targets. Those investigators reported direct selection for sensors which is simplified as described herein, reflecting previous results with competitive sensors. In this variant, the ability to of library members to survive cycles of selection and amplification is connected to their ability to interact with steroids in a way it competes with an oligonucleotide complementary to the primer region (FIG. 2a). This complementary oligonucleotide is used to capture library from the solution, and is then displayed on an affinity column.

These conditions provide the ability to immediately study binding via fluorescence sensing. Taking advantage of this benefit dictated that one of the stems would not randomized, as the potential for full binding to competitor oligonucleotide in the vicinity of the binding site is preserved. Otherwise, the selection conditions lead to the preferred release from the column due to shorter complementarity (weaker binding) with the competitor oligonucleotide.

A selection buffer with very high salt concentrations was selected for the work described herein, in contrast with the previous work. This choice reflects the need to overcome the variability in salt concentrations of urines, and its consequence is that some steroids will be less soluble, and some may have changed binding to hydrophobic three-way junctions (e.g., via “salting out”).

In this experiment a counter-selection was not performed. First, hydrophobic receptors cannot be fully specific, and even if selectivity over one steroid could be achieved, there would be cross-reactivity with other. Also, sets of two or three specific sensors would be unable to detect more than, respectively, two or three analytes. Finally, without standardization against creatinine, specific sensors would be useless in urine, while cross-reactive sets would provide us with a characteristic pattern. However, even in light of the foregoing, certain embodiements of the present invention can benefit from counter-selection. In addition, the stringency of the counter-selection can be modulated such that even in the instant context a counter-selection could find use.

### 1.2. Selection Process

Selections were performed with four different steroids (FIG. 2b), representing large families; three were of actual diagnostic interest (DOG DIS, and DCA) and one was primarily of a structural interest (β-estradiol, BE). Estradiol has a different core and not identified a single sensor that would prefer it over other steroids has previously been reported. Further, the classification of DOG and DIS over a range of concentrations with unmodified junctions was traditionally the most challenging for small arrays, thus isolation of a pair of sensors that can do this effectively would be of practical significance. Concentrations of steroids in selections were set below concentrations typically present in the urines of patients. Thus, selection on sensors that will be diagnostically useful was the instant focus, with urine being diluted with buffer.

First selections and optimizations of conditions were implemented in parallel with DIS SELEX and DCA SELEX, with cloning performed after six and eleven rounds in the former case. In two other selections cloning was performed after 9 rounds for estradiol and 13 rounds for DOG SELEXes and confirmation via semi-quantitative PCR that significant amount of DNA is released from an affinity column upon exposure to steroids. After each cloning, snapshots of the pool were obtained by sequencing and analyzing up to 24 clones.

Cloning and sequencing was accomplished as follows. During in vitro selection step, every fraction was collected to monitor the elution profile of SELEX via PCR. After obtaining the clear elution profile which showed a big increase by target addition, a cloning step was carried out. To prepare the insert DNA for cloning, PCR amplification was carried out in the same PCR condition as aforementioned, except the final extension step. The sample was incubated 15 min at 72° C. in final extension step to add A-tail sufficiently, to use T/A clone system. The amplicons were purified as same method mentioned above and the purified products were directly incorporated to plasmid vectors using TOPO TA Cloning kit (Invitrogen) following the manufacture's instructions. The plasmids were subsequently transformed into OneShot TOP10 competent cells (Invitrogen), positive clones containing recombinant plasmid DNA were screened via blue/white screening. Approximately 20˜30 colonies were picked in each cloning and the plasmids were isolated using PureLink Quick Plasmid Miniprep Kit (Invitrogen). Positive clones were confirmed by PCR and sequencing was performed by the standard DNA sequencing service facilities (https://www.dnasequencing.hs.columbia.edu/). The analysis of electropherograms files (ABIfiles) and verification of each error were completed by Chromas Lite (http://www.technelysium.com.au). The multiple sequence aliments were carried out using CLUSTALW (http://www.ebi.ac.uk/Tools/msa/clustalw2/). From the 6 time cloning, total 149 sequences could be obtained and 101 sequences forming 3-way junction type among them based on the secondary structure analysis (mfold, http://mfold.rna.albany.edu/) were found.

Among the predicted three-way junction sensors based on mfold analysis, 4 steroid sensors; S-Sensor 1, S-Sensor 2, S-sensor 3, and S-Sensor 4 were selected.

All measurements were performed in the 1×SELEX reaction buffer with the indicated steroid concentration. Mixture of sensors and Q-capture strand was incubated 5 min at room temperature, and then a series of standard dilutions of all compounds (the stock solution of each compound was adjusted to pH7.4) were added to the mixture solutions to final concentrations of 50 nM sensors and 150 nM Q-capture. The mixtures were incubated for 30 min and transferred on 384 well non-binding surface, flat bottom, black polystyrene assay plates (Corning, N.Y., USA), then the fluorescence was measured with a 485-nm excitation filter and a 535-nm emission filter by using a Perkin-Elmer Victor II microplate reader (Shelton, Conn., USA). The background fluorescence signal (BFS) for each batch of measurements was taken as the average of 1.0 concentration fluorescence readings (without analytes) for each of those measurements. Then, each fluorescence measurement was normalized by dividing by the responding BFS for that analytes at that concentration.

### 1.3. Analysis of Receptors/Sensors Isolated in the Selections

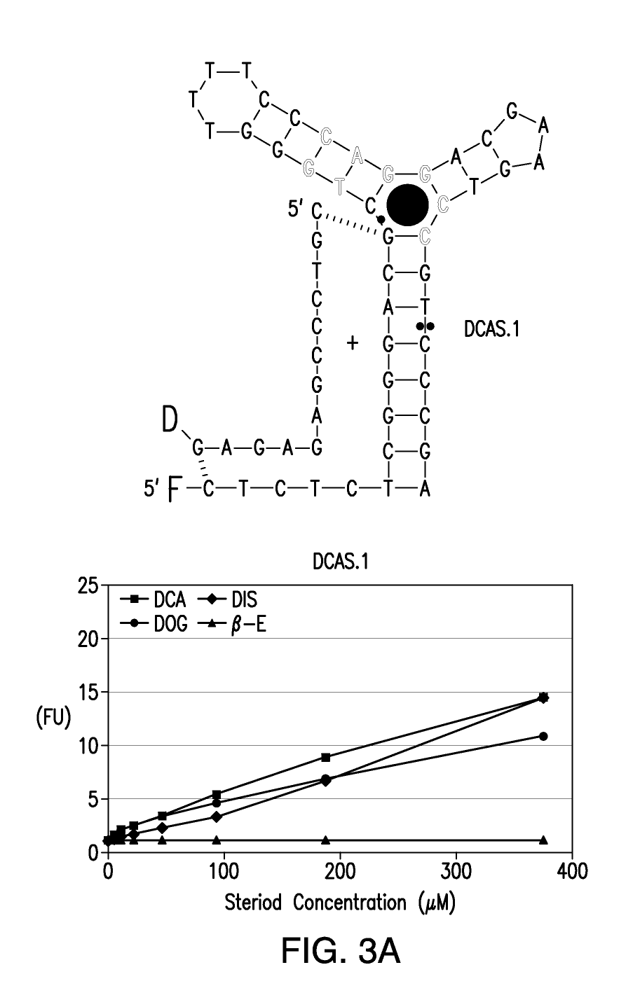

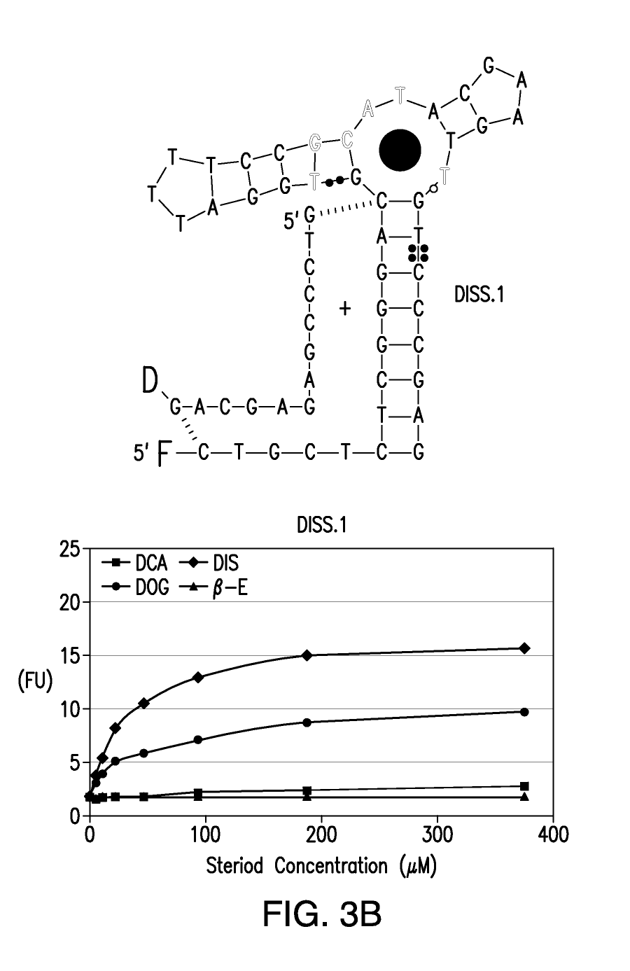

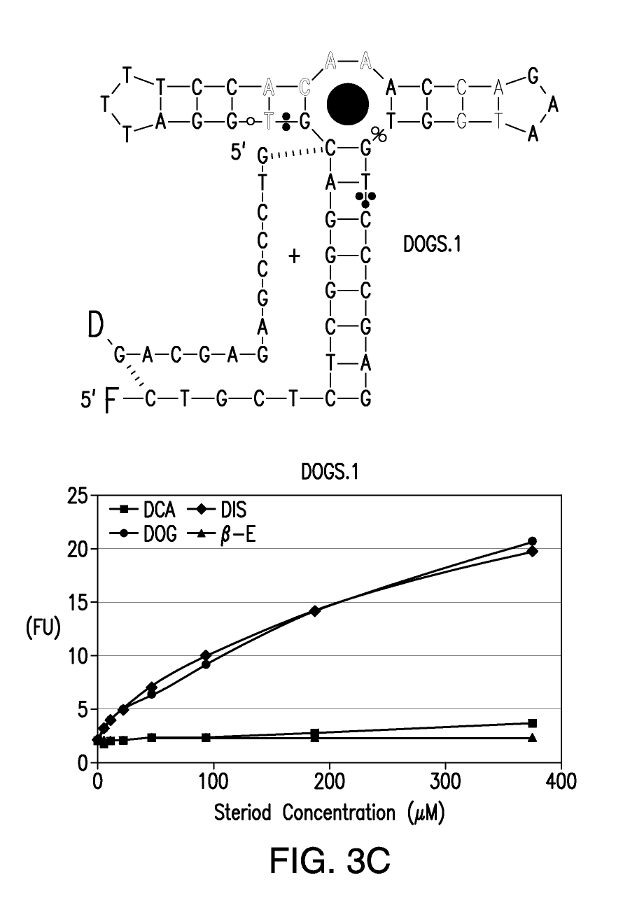

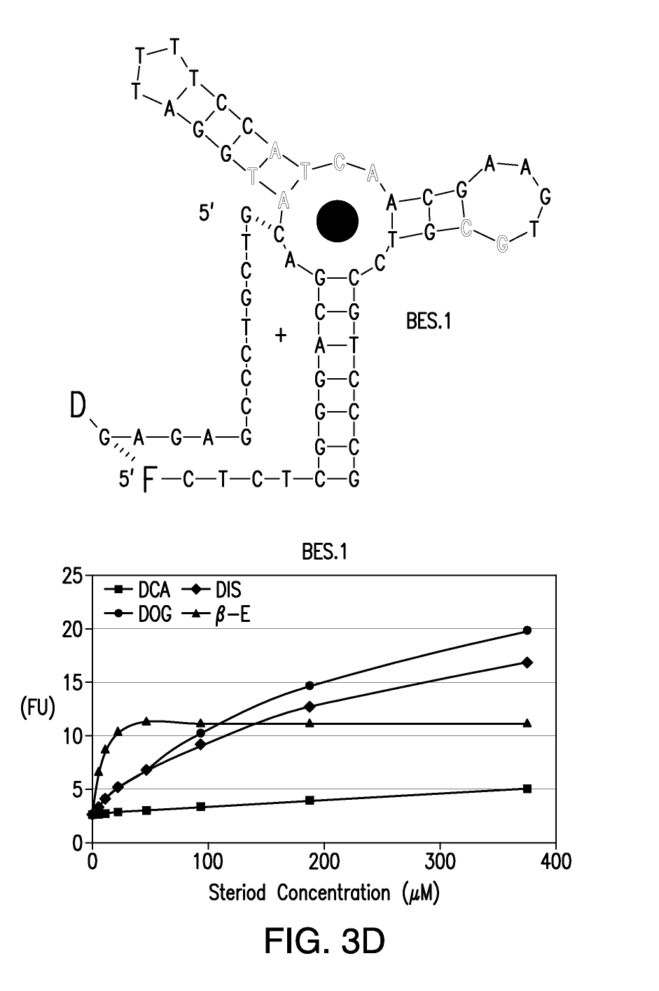

The identified sequences from individual selections were ordered according to their observed frequency and tested. The selection was designed with the presumption that the most frequent sequences will be those most strongly interacting with the selected classes of steroids. Analysis and testing of the sequences indicated that the actual selection pressure was result of the balance between interactions with steroids and reduced binding to complementary oligonucleotide on the affinity column. As the result, only one out of four of the most abundant structures from four selections could be completely mapped to the initial library as ordered, while other many sequences had missing bases (e.g., in DOG SELEX up to seven, FIG. 3c). Further, none of the most abundant sequences had folded according to the initial proposal (including fully-matched structure from DCA SELEX), although all of them found a way to form some variation of the three-way junction. Some sequences had also unexpected mutations, presumably results of errors during PCR-amplification, while some even had extra bases.

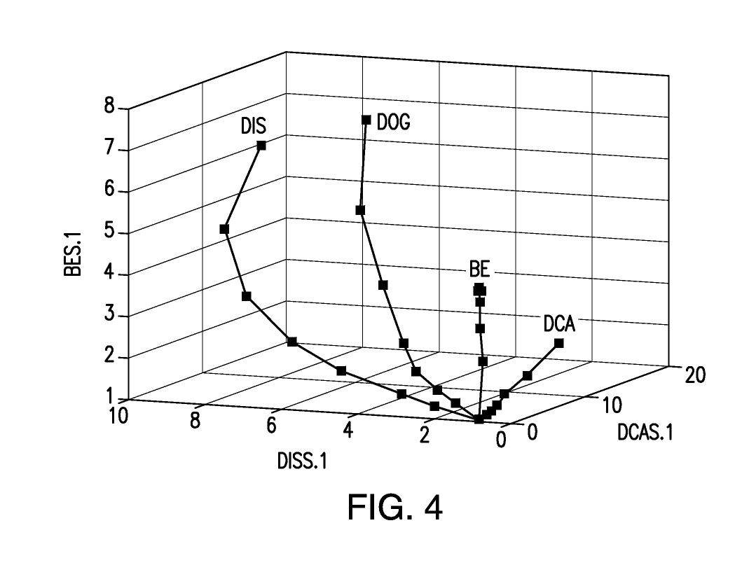

After further optimization of structures to improve sensing, four sensors were selected, representing the most abundant aptamers from each selection, as shown in FIG. 3. These four sensors did not fit the “best-case scenario” from the design, with inversions of selectivity, but were clearly differentially cross-reactive. As predicted in the initial design considerations, these four sensors allowed us to classify pairwise analytes at all concentrations by projecting them separating them in 2D. Also, three sensors were sufficient for perfect classification of these four analytes (FIG. 4), even though they had no modifications in their binding sites.

Overall, although the results were not exactly what was expected based on the input library and the receptor space did not provide in all cases receptors with inverted selectivity, the evolutionary procedure resulted in very satisfactory array.

### 1.3. Comparison of Sensors and Receptors to Previously Known Hydrophobic Receptors

Selection for binding to deoxycholic acid served as a positive control. Koto and coworkers previously demonstrated that three-way junctions are optimal receptor for this class of steroids, and their results were confirmed with the sensors as well; DCA SELEX resulted exclusively in the fully matched three-way junction type sensors.

Other three steroids, however, yielded unexpected results, as none of the most abundant sequences was tested in previous manual screening. These results indicates the advantage of evolutionary approaches in identifying junctions with multiple differences from known binding motifs. BES.1 with the highest selectivity for estradiol represents a completely new junction motif with extended linker regions connecting hydrophobic surfaces; nothing similar was even considered for manual testing, as there was no rational reason to propose testing such a design. Random individual testing of expanded junctions resulted in presumably “collapsed” hydrophobic pockets, that is, junctions with no response to steroids.

Junctions DISS.1 and DOGS.1 are similar, but not identical, to junctions previously tested, but resulted in no satisfactory results; e.g., nonresponsive variants with two T's in a linker (vs. two A's in DOGS-1) and a variant with T instead of A in DISS-1 were tested. These results again showcase the advantages of evolutionary searches, allowing an exhaustive access to sensors.

In this report the following advances are offered: (i) From the perspective of hydrophobic receptors and sensors, the richness of three-way junction space is demonstrated and, more generally, the surprising variability of hydrophobic receptors space that is accessible to nucleic acids. Similar classification results could only be achieved using prior techniques by employing modified junctions and non-junction sensors. (ii) From the perspective of the practical cross-reactive arrays, a straightforward evolutionary search procedure was established that, being performed with an analyte in the solution-phase, is suitable for any classes of hydrophobic compounds, including actual patients' samples. (iii) From the perspective of basic science of cross-reactive arrays, it was confirmed that minimal sets capable of perfect classification could be built bottom up, through assembling suitable sensors isolated through interactions with individual classes, completely eliminating complex data analysis. In contrast, prior techniques employed top down methods, starting from larger collections of junctions, using manual screening followed by machine-learning techniques to trim down initial sets of sensors by studying classification powers of subsets. And, (iv): partially randomized libraries with preexisting structural motifs were employed; this method is asking to a knowledge-based pre-filtering, a method used to reduce the search space in the data mining field. The potential cost of this approach is that there may be better individual sensors which could be identified through broader search.

In conclusion, the direct connection between genotype and phenotype in certain embodiments of the instant receptors enabled application of evolutionary methods to mine rich junction space for responsive junctions and rapid assembly of arrays of sensors.

### Example 2

### 2.1. Evolutionary Selection Through Training of Minimal Cross-Reactive Arrays

This example is based on a straightforward, yet novel idea based on the availability of a set of receptors (TWJs) amplifiable by PCR that has numerous members that can interact differentially with steroids. Steroids are grossly changed in wines of patients with metabolic diseases. Thus, an evolutionary search for receptors in this set that preferentially interact with the dominant steroid component common to all urine samples coming from patients with a particular metabolic disease can be employed (class [DISEASE], e.g., positive subset of “FREE CORTISOL”). Receptors that fail to interact strongly with all members of these sets will be suppressed in comparison with receptors that do interact strongly with all members. This selection can be repeated to evolve the other group of TWJs that interact with what is common for class [HEALTH]. The union of two evolved sets and, in particular, the best represented unique members (i.e., those that survived and multiplied) will be useful in diagnostic arrays.

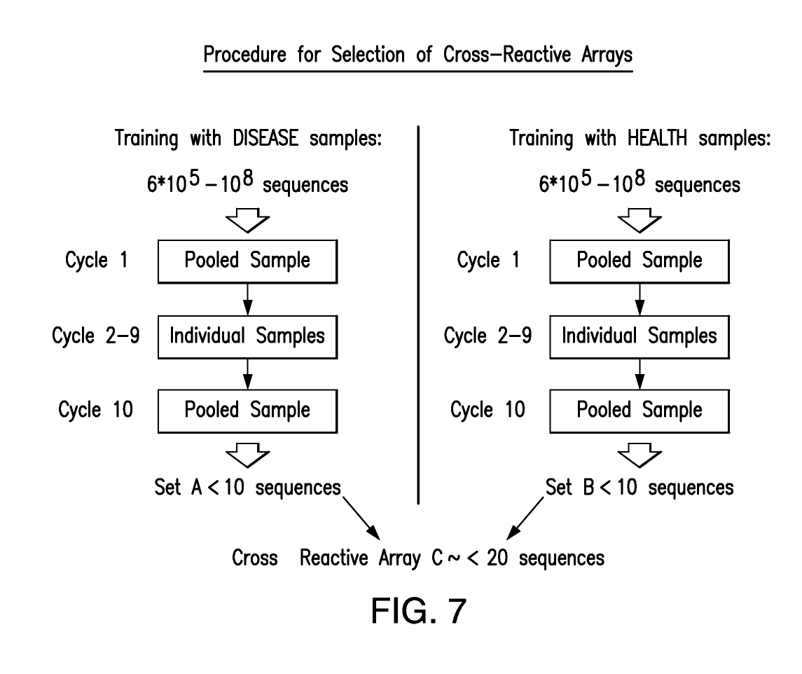

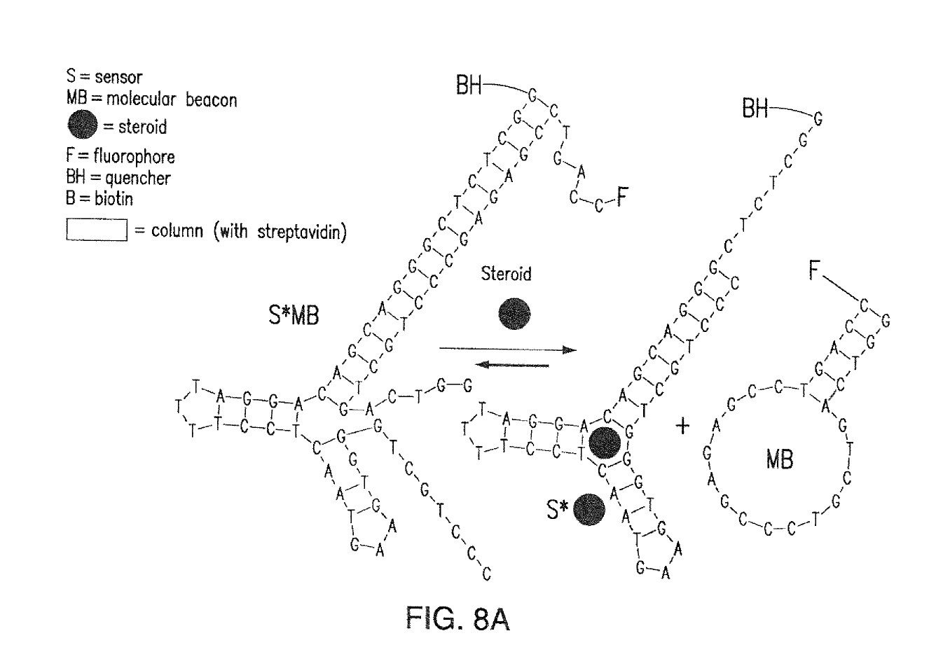

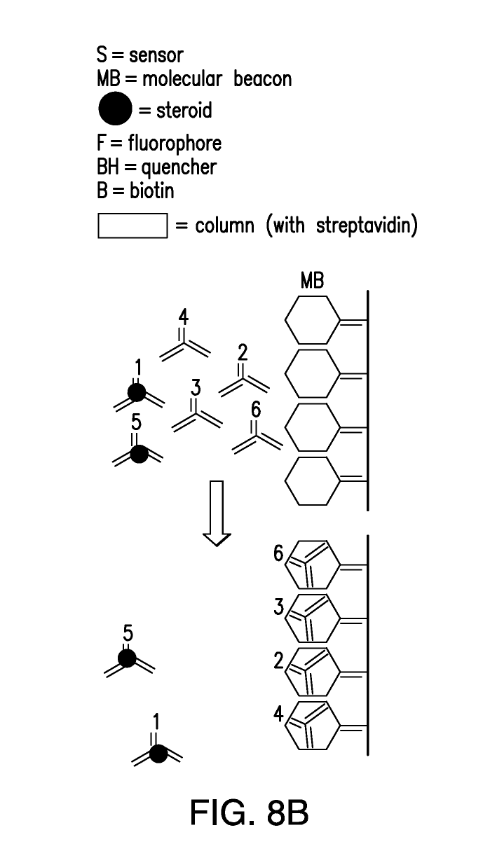

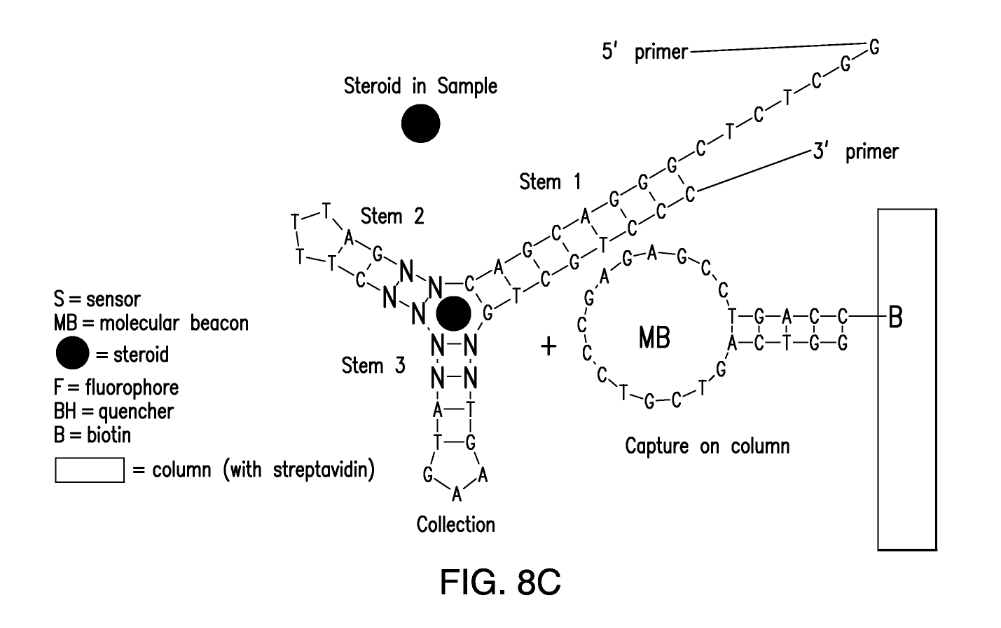

FIG. 8b explains in some detail the initial selection of unmodified and symmetric junctions. To avoid confusion the remainder of this example assumes “FREE CORTISOL” training samples; the same procedures (perhaps with different modified nucleotides) can be performed for “LIVER FAILURE” set and with modified nucleotides. The experiment starts with the collection of oligonucleotides that will be selected for the ability to bind steroids (FIG. 8b). The collection comprises 48 junctions (˜6.6*10), i.e., an oligonucleotide library (FIG. 7c) with eight randomized positions (N8; the size of the library can be readily increased to 107 or more members). Due to the pseudo-symmetry within junctions, this collection can be reproduced on several microarrays (˜4, with controls) and the progress of the selections can be monitored in parallel. Many alternative PCR-amplifiable libraries are available. For example, 10 bases can be randomized, or positively charged dT analog can be used, or hydrophobic groups can be put on a primer. Three of these libraries will be tested in this example.

The starting point in selection is ˜1,000,000 copies of each receptor, but some are damaged due to the synthetic nature of the library (these damages disappear after PCR). Individual junctions are in the form that allows steroids to compete (displace) with an oligonucleotide competitive inhibitor (a stem loop form is used as a competitor oligonucleotide—MB, from molecular beacon, FIG. 8a). This is a variation of structure switching sensors. Junctions that are incapable of reacting with MB can be eliminated by filtering the collection through a column displaying MB, followed by the displacement of the junctions captured by MB by the full complement of the MB. The length of MB can be optimized to minimize the spontaneous release of the junction per column volume elution.

The starting collection of training samples will have two classes, e.g., eight samples in [INCREASED CORTISOL] class and eight negative samples in [HEALTH] class, all buffered with 20×[TRIS, pH 7.4, with 10 mM MgCl2, and 2 M NaCl], with no other pretreatment. This buffer will overwhelm any individual variations in samples. SELEX begins with pooled eight positive samples, guaranteeing that the initial choice of the individual sample would not completely eliminate favorable library members, due to some idiosyncrasy in one sample. A further eight cycles of training, using individual patient samples, proceeds as follows.

The library is incubated with the first buffered training sample and passed over the column containing MB (FIG. 8c). This format is the opposite from SELEX that was used in the art to isolate aptamers in structure switching format). All junctions that are strongly responsive to the sample will be weakly captured by the column, and vice versa. Thus, the selection is Darwinian and for survival of a “culling” procedure that eliminates the unfit junctions. The selected junctions are precipitated and PCR amplified. After eight individual training sessions, the process can be repeated with the pooled class and proceed to identify 100 surviving sequences (cloning and sequencing procedure). This collection of 100 sequences will provide a distribution of junctions that significantly interact with samples of [INCREASED CORTISOL] class and set A can be defined as junctions representing more than 10% of the clones (fewer than ten sequences and the selection stringency was 10%—these can be easily adjusted).

Next, this procedure is repeated with [HEALTH] samples obtaining 100 sequences to define set B, with junctions interacting with [HEALTH] samples. The union A&B of the two collections of junctions can be identified (see FIG. 5C), resulting in set C of junctions, which is a cross-reactive array classifying the samples into categories. This array is then minimized to three-sensors using the training collection of samples. Cross-validation is then performed with individual training samples. This would be followed by a blind study with validation sets.

In conclusion, to explain in a somewhat simplified fashion, why this SELEX will work and produce directly excellent cross-reactive arrays: the sample classes, [DISEASE] and [HEALTH] with their different compositions of steroids, produce different distributions of junctions that are responsive, subsets A and B. Once they are combined into set C, they are exposed to an unknown Ω, for example, belonging to a [DISEASE] class. Then, within set C, junctions from the subset A & NOTB will be more responsive to a than junctions from the B & NOTA subset. If Ω belongs to a [HEALTH] class, the reverse will be true. This is so by definition, because of the way set C is constructed and is also by definition a differential sensing, as demonstrated in FIG. 6. It is possible to have a situation in which NOT(A&B) is an empty set, however, excellent resolution is possible if there is significant difference in distributions (weights) between individual junctions in sets A and B (FIG. 2). The stringency can be decreased and the junctions combined at stringency of 5% level, eliminating junctions that have the same weights (distribution percentages).

The rationale for the experimental design is different from a typical SELEX procedure and almost natural reaction to the selection is the following question: Why not perform subtractive SELEX, and remove junctions belonging to the set B, using junctions that belong to the set A & NOTB?First, it is likely that the same steroids are present in both sets, but at different ratios; this would lead to problems with urine dilutions in individual patient samples; it would not be known whether the high concentration is a sign of a disease, or just of concentrated urine. To distinguish these two cases, a pattern is needed.

Second, it is most likely that even if there is a highly specific compound, e.g., for immediate differentiation of Cushing syndrome in all patients, it would be difficult to find a specific junction for it, because steroids are very similar and junctions are highly cross-reactive. And while a particularized detection method has its uses, a general method, applicable to all classes of diagnostic samples, would also be useful.

The first potential problem is that statistically indistinguishable distributions of junctions in both sets A and B may be observed, even at the 5% cut-off level. Thus, the new combined array would have very poor classification power. The likelihood of this problem is proportional to the variability that exists in the junctions—and the first collection has the smallest variability. This issue can be addressed by increasing the size of the pool (e.g., N14) and variability through introducing new functionalities in the junctions. Procedures starting with an aminoallyl-dU derivative (switching to non-thermocycling polymerase to generate the library for SELEX; this analog has been used earlier in a selection for a catalyst) can be used with increased resolution for negatively charged conjugates. Additional hydrophobic surfaces close to the binding pocket can also introduced into the primer. Results of two different selections can also be combined and selections at approximately one cycle per day can be run. Also, Section 2.2, below, represents an alternative strategy, allowing the inclusion of junction sites with non-amplifiable modifications.

The second potential problem is, to a degree, opposite from the first: the possibility that no convergence of the junctions is observed. This problem is addressed by increasing the stringency of selection, e.g., diluting urine samples, eliminating elutions from selection, reducing the pool, or increasing the competitor size.

Existing sensors are known to respond as expected to the additions of standard steroid in urine, thus, problems once “best” sensors are identified are not expected, however it is conceivable that some unknown impurity in one or more samples may interfere with selection. This can be addressed by focusing the selection on the hydrophobic (steroid) fraction of the urine by first using reverse solid-phase extracts of urine (isolating on C.18 column hydrophobic, that is, steroid fraction). This is expected to lead to more rapid convergence of the pool and avoidance of idiosyncrasies in urine samples. Also, after this selection, actual components of urine that interact with individual sensors can be isolated and identified, using affinity selection/LC-MS. This would assist in understanding the arrays, and perform trouble shooting, if there is an interfering component. Importantly if there is an interfering component, direct counter-selection at the concentrations that are expected in urine can be run.

The evolutionary search will produce up to three arrays for each of the training sets “FREE CORTISOL” and “LIVER FAILURE” (up to total of six arrays if different healthy controls are used). Arrays will be based on unmodified junctions, positively charged dU derivatives, and one of the hydrophobic derivatives at the primer. Based on the stringency criteria, up to 6×20 sensors may be used, but a significantly lower number is expected, because of the likely overlap in junctions (minimum is 30). This set can be expanded by sensors previously isolated for detection of alkaloids and steroids (“120+” set).

Specifically, each junction can be turned into a solution-phase sensor form. The selection produces immediately recognition part of sensors, and the actual sensors are constructed by adding fluorophores. Because of the background fluorescence of urine, Cy5 dye and Black-Hole 2 quencher will be used. Each sensor will be measured against each of the samples from both training sets in triplicate. This work will be done in a high-throughput facility using Perkin-Elmer cherry-picking robots suitable for exactly this type of work. Sensors will be individually synthesized and purified by IDT DNA and each sensor will be characterized by CE to ensure over 90% purity (total cost of synthesis is estimated at $25,000). Additional samples can be generated by mixing individual samples, if needed. These results can be organized in a crossreactive array with 120+ sensors with each sensor reporting measurements for 36 samples in triplicates and in two dilutions (2×2×8 from training sets, and 4 pooled measurements from each class, with positive and negative controls less than 30,000 measurements or approximately 80 384-well plates).

In prior work, 11 sensors were used to perform measurements of 15 alkaloids/steroids over a range of concentrations (total of −7,000 measurements). Then results from all possible subsets of 11 sensors (2-10 sensors) and performed leave-one-out (LOO) validation using k=3-7 nearest neighbor classifiers. The results are used to define the minimum array with maximal classifying power, also maximizing the number of individual classes that sets of 3 sensors can classify. For example, a three-sensor (3D) array classified perfectly (99.57+/−0.43%) ten alkaloid/steroid analytes over the range of concentrations limited by analyte solubility and sensor sensitivity. Because the analogous exhaustive search procedure is computationally difficult with 120+ sensors, thus, it can be advantageous to focus the search on those subsets of 2-5 sensors that can accomplish >95% classification of samples within the original training set, and over 90% classification within combined training sets. The union of three best arrays (<10 sensors) will be combined into a candidate array which can be further validated as outlined in Section 2.3, below.

### 2.2. Screening of Junctions Using Microchips

A recently developed microchip technology (the word ‘chip’ is used instead of ‘array’ to avoid confusion with cross-reactive arrays) provides an opportunity to screen at once a large number of variations in structures, particularly when the nature of the analogs prevents us from running SELEX. This example has two main goals: First, develop a back-up method for the SELEX; second, allow for an increase in the resolution of junction space with non-PCR amplifiable oligonucleotides. The latter goal is even more important in the long run, because of the targeting of other metabolic diseases.

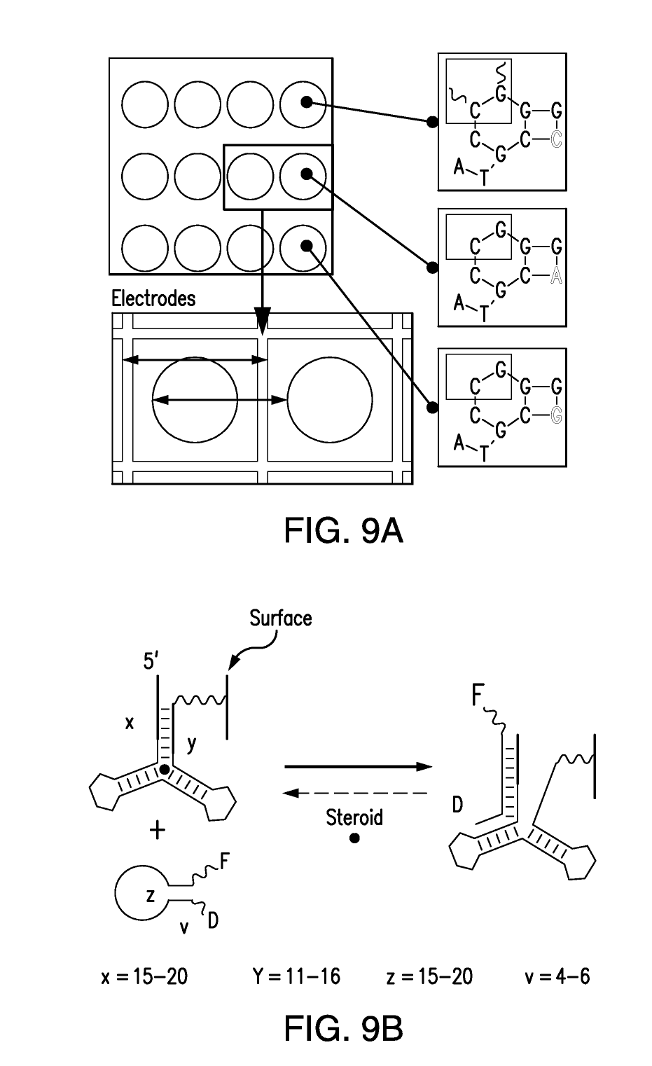

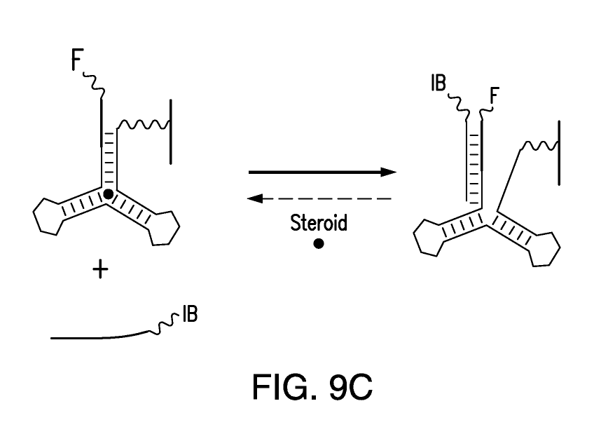

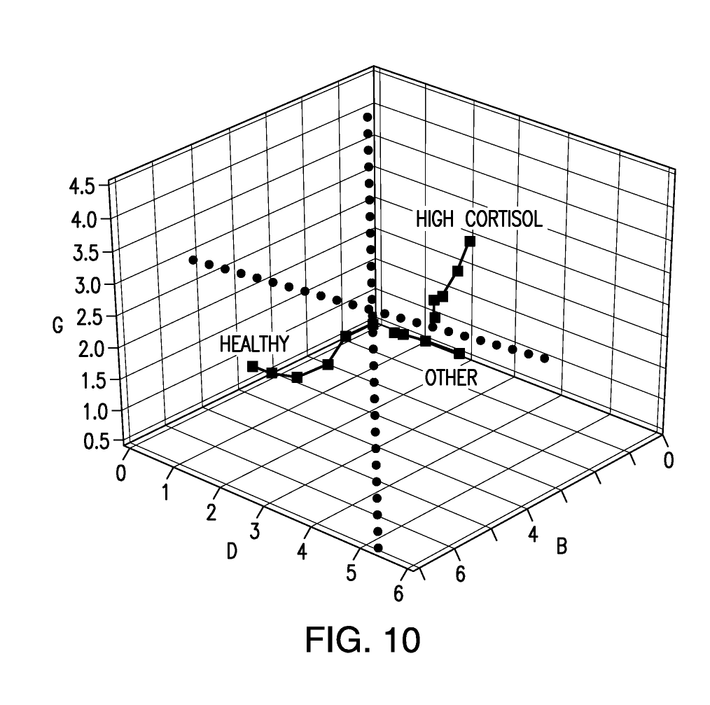

We will use (formerly) CombiMatrix Inc. microchip technology (i.e., CustomArray, now sold by Custom Array), in which the 3-to-5′ synthesis of oligonucleotides (reliable up to 50-mers, although the sensors are shorter than that, e.g., S in FIG. 5a is 45-mer) is performed on electrically addressable spots on a microchip (in reality electrodes 44 μm in diameter with the distance between centers of two neighboring spots 75 μm, cf., FIG. 9a; the synthesis is based on phosphoramidite chemistry and occurs at all sites simultaneously according to a computer algorithm that activates specified electrodes. These electrodes are actually removed from oligonucleotide probes, and serve to electrochemically detritylate intermediates, through a pH increase. This technology provides us with the ability to program the parallel synthesis of, e.g., 12000 oligonucleotides (the 12K chip).

The results on chips are conventionally obtained using fluorescence detection, indicating no problems in quenching of fluorophores. The data provided by the company indicate that the average pair-wise correlation coefficient (r2) among different sectors on the same microarray was 0.97-0.99 for fluorescent detection, with cv's below 4-11%, and error rates in oligo-detection of 0.15%. These arrays have previously been used to study sequence space of aptamers.

The microchip assays are more complicated than solution-phase assays, due to diffusion and surface effects, cross-contamination issues, and more complicated experimental procedures. Assays for microchips will be optimized and validated on six representative steroids (corticosterone, dihydro-isoandrosterone, deoxycholic acid, 16-hydroxy-pregnenolone, 15β,17-dihydroxypregnanolone, and estradiol-17-sulfate) at 20 μM concentrations in urine depleted of steroids through a solid-phase extraction and on four sensors that are responsive to these steroids.

The assay will first be tested on a 1K chip with eight cross-reactive junctions that bind steroids and four that bind poorly. 840 spots will be used for these junctions, 7×10 for each TWJ, in order to establish the reliability of the method. 80 spots spread throughout the chip will be used as positive controls for binding (e.g., a complementary oligonucleotide with no junction, and 80 will be used as a negative control to establish background fluorescence. These will be used to normalize the signal changes in individual junctions. This initial testing may be repeated to optimize the competitor oligonucleotide

One assay can proceed as follows: a molecular beacon (MB, cf. stem-loop in Section 2.1, above, but with Dabcyl and Cy5, FIG. 9b) is constructed that interacts with the junction at the 5′ end (the beacon needs a longer interaction complementary region than the open competitor). The 5′ terminus will be identical for all junctions and one beacon will be sufficient per chip. The chip will be exposed to the beacon, first alone, then in the presence of a steroid sample and the relative decrease in fluorescence on the spot in comparison with controls reflects the strength of interactions with the steroid. Using red dyes (e.g., Cy5) and a highly-purified stable beacon will minimize the urine fluorescent background and allow for skipping the washing step. It is expected that some cross-contamination of the spots on the junction will occur, strongly reactive junctions will release oligonucleotides that could rebind to the weakly reactive junctions. In this case spots with an increase in fluorescence will become even stronger when contrasted to spots that decrease in fluorescence and this “rich-becoming-richer” situation increases the ability to differentiate junctions.

The beacon-format of an assay has several advantages: First, the chip is less expensive because it does not involve adding fluorophore during synthesis, more than offsetting the increase in a beacon's cost. This allows for one more modification within junctions on the chip to be used. Second, photobleaching is not an issue, as fluorescent reagents are always renewed. Third, the chip can be regenerated under very mild conditions and multiple times by a beacon complement, eliminating stringent conditions used for the removal of probes by the chip provider. It could be argued that the negative aspect of the assays is that fluorescence decreases with binding, but the screen will be at high concentrations of steroids and sensitivity is less of an issue. Note that Cy5 does not overlap with urine fluorescence and that that communication with company representatives indicates that fluorescence is conventionally used to assess binding to these arrays.

Alternatively, the junction in structure-switching format capped at the 5′ end with a fluorophore (F in FIG. 9c) will be exposed to a quencher-labeled competitor oligonucleotide (Iowa Black or Black Hole 2 with steroids present in the solution. While this assay has the advantage of producing an increase in fluorescence in the presence of steroids, it does not allow for the use of the chip as many times due to eventual photobleaching (although standardization against a positive control, switching to the less bleaching-prone fluorophore, and use of oxygen scavengers in the solution would help).

A fluorophore-labeled competitor can be used in solution in a two step assay (this procedure can be demonstrated with flow-cytometry and beads). First, the unlabeled stem-loop competitor is displaced from responsive junctions. The oligonucleotide binding site is now unoccupied, and a new batch of a fluorescently labeled competitor oligonucleotide (same or without the stem, to increase the reaction rate) will prefer to interact with junctions occupied by steroids. These will become more fluorescent, as the steroid is competed out (competitor is present in an excess, and urine solution is removed), while junctions with oligonucleotide competitors will remain dark. While this is a more complicated procedure (increase in noise is likely) and would require longer optimization, its advantage is that increased steroid concentration is translated into an increase in fluorescence, while photobleaching is still avoided.

After unmodified junctions, junctions with short (C3) spacers, aa-dU, nitroindoles, and pyrenyl analogs—all available from Glen Research can be used. Single modified nucleotides (including spacers) can be introduced in CombiMatrix synthetic procedures with only nominal price increases. On the example of spacers: various active junctions from the first screening can be used to construct a chip in which one, two, and three spacers (or analogs) are introduced into the junctions. For all junctions seven different positions will be tested in which spacers can be introduced in the junction (there is one way to put three spacers in a junction, and three ways to put one and two spacers each), and this can be further increased by putting spacers next to the junction (i.e., as a mismatch).

Junctions that will have high response in each of the steps of the training procedure are available, and these can be identified and pooled to obtain trained array through trivial data handling procedures. More interesting aspect is for this array to be compared to the selection procedure in Section 2.1, above, and a set of clones from each step of the SELEX can be used for comparison. This would allow for troubleshooting of SELEX (e.g., discover why SELEX is giving different results, if some junctions are poorly amplifiable or similar), but also vice versa: Once SELEX is successful, it could be mimicked with a simulation that uses microchip data (e.g., using Darwinian search algorithms). This will lead to in silico/on-the-chip SELEX with modified nucleotides, in which a computer program will provide “weights” (“survivability parameter”) to individual junctions. The weights can be adjusted for the result to match SELEX, and then applied for future selections, with non-amplifiable primers.

At the end of this procedure, one junction from each of the subgroups (˜20 or less subgroups, after removing redundancies are expected) will be taken and used to perform further dimensionality reduction via cross-validation, as described previously, but with patient samples.

Some standard issues with microarray optimization and concerns with noisy data were addressed earlier. Prescreening chips, in order to eliminate junctions that give poor signal with the competitor oligonucleotides may be useful. Another potential problem is too many positive junctions could be identified, in which case an increase and optimization of stringency (e.g., relative fluorescence change leading to selection of junction, dilutions of urine, washing, blocking non-specific interactions, etc.) will be performed. Also, the structures can be re-checked, and assessed as to whether the hits are real—by synthesizing junctions in solution. Finally, if urines end up being problematic for some unexpected reason, solid-phase C18 extract of hydrophobic/steroid fraction can be used as described above. Again it is noted that sensors keep their response to steroids in urine, and the reason for this extraction would be to avoid possible problems stemming from any incompatibility between urine samples and microarrays that might be uncovered.

### 2.3 Minimization and Validation of Arrays for Steroids

Samples of “FREE CORTISOL” and “LIVER FAILURE” can be further separated in two groups: a training/minimization set with of samples, and a validation set with % of samples. The training/minimization set will be used to first generate subsets of 3-5 sensors from the initial group of 20 sensors, with the ability to classify these samples in [INCREASED CORTISOL], [INCREASED BILE ACIDS], and [HEALTHY], with a fourth class, [OTHER] also possible. The response of each sensor to each urine sample will be measured individually and in triplicate. The result may be one array with three sensors that is, for example, 90% accurate in classification for both diseases, and many arrays with even only two sensors that have 99% accuracy for individual sets. These results will allow us to clearly determine Euclidean distances (and sensor ratios) for decision trees to be used for the next and last step in the strategy.

As the final result of this minimization, one or two optimal arrays will be generated each consisting of two-three sensors. These arrays should be able to classify the samples in validation set, and their error rates in classification using the k-nearest neighbors method against the training set will be determined. The samples will also be characterized in 3D space and decision trees with all outliers further characterized. For example, some common medications that cause unusual shifts in positions may be identified. For cortisol samples it will be checked whether there is a distribution that would correlate with the source of Cushing. It is expected that this will be case, because the source is reflected in urine as changes in ratios of 17-KS and 17-OHCS.

Using the techniques of the present invention, it is possible to assess the classification error rates of optimized arrays, and their ability to classify patients' samples into [INCREASED CORTISOL], [INCREASED BILE ACIDS], and [NOT INCREASED] classes, and [OTHER]. [INCREASED CORTISOL] will be further stratified into subclasses based on the sources of Cushing, and the size of the study will allow us to perform LOO cross-validation and obtain statistical significance of a “diagnostic classification”. Samples that stand out or that are misclassified will be first repeated (to avoid experimentalists' error), and in the case of confirmation will be separately characterized at several dilutions and tested for possible interferences; they may be further sent to other steroid/drug tests. At the end of this process, the diagnostic potential of the sensor arrays can be understood, and further clinical evaluation and expansion of the approach outlined herein can be undertaken.

Various publications are cited herein and listed in the attached Reference List, the contents of all of which are hereby incorporated by reference in their entireties.

### REFERENCE LIST

1. Albert et al., 2000. Cross-reactive chemical sensor arrays. 2595-2626.
2. Roeck et al., 2008, Electronic Nose: Current Status and Future Trends; Chem. Rev. on line, thematic issue. DOI: 10.1021/cr068121q.
3. Lavigne et al., 2001. Sensing a paradigm shift in the field of molecular recognition: from selective to differential receptors. 3118-3130.
4 Wright et al., 2006. Differential receptor arrays and assays for solution-based molecular recognition. Chem. Soc. Rev. 35, 14-28.
5. Francois Jacob in The Possible and the Actual.
6. Stojanovic et al., “Self-assembling aptameric sensors” J. Am. Chem. Soc. 2000, 122, 11547-11548.
7. Stojanovic et al., 2001. Aptamer-based folding fluorescent sensor for cocaine. J Am Chem Soc 123:4928-4931.
8. Stojanovic et al., “Aptamer-based colorimetric sensor for cocaine” in J. Am. Chem. Soc. 124: 9678, 2002.
9. Guo et al., N. R. 1989. Site-specific interaction of intercalating drugs with a branched DNA molecule. Biochem. 28 (6): 2355-2359.
10. Lu et al., 1992. Interaction of Drugs with Brached DNA Structures. Crit. Rev. Biochem. Mol. Biol. 27 (3): 157-190.
11. Kato et al., 2000. In vitro selection of DNA aptamers which bind to cholic acid. Biochim Biophys Acta 1493:12-18.
12. Kato et al., 2000. Interaction of three-way DNA junctions with steroids. Nucleic Acids Res 28:1963-1968.
13. Oleksi et al., 2006, Molecular Recognition of a Three-Way DNA Junction by a Metallosupra-molecular Helicate. Angew. Chem. Int. Ed., 45, 1227-1231.
14. Stojanovic et al., 2003. Cross-reactive arrays based on three-way junctions. J Am Chem Soc 125:6085-6089.
15. Green et al., “Rational Approach to Minimal Cross-reactive Arrays” J. Am. Chem. Soc. 2006 (128) 15278-15282.
16. Pei et al., “An approach to high resolution cross-reactive arrays based on matching binding motifs” Chem. Com., 3193-3195, 2009.
17. Ellington et al., In vitro selection of RNA molecules that bind specific ligands. (1990) Nature (London) 346, 818-822
18. Tuerk et al., Systematic evolution of ligands by exponential enrichment: RNA ligands to bacteriophage T4 DNA polymerase. Science. 1990 Aug. 3; 249(4968):505-510.
19. Robertson et al., Selection in vitro of an RNA enzyme that specifically cleaves single-stranded DNA. Nature 1990, 344; 467-468.
20. Dill et al., Immunoassays based on electrochemical detection using microelectrode arrays. Biosens. Bioelectron. 2004 Nov. 1; 20(4):736-42.
21. Dill et al., Antigen detection using microelectrode array microchips Analytica Chimica Acta. 2004: 69-78.
22. Liu et al., Integrated microfluidic biochips for DNA microarray analysis. Expert Review of Molecular Diagnostics. 2006; 6(2): 253-261.
23. White, W. I. A New Look at the Role of Urinalysis in the History of Diagnostic Medicine. Clin. Chem. 1991, 37/1, 119-125.
24. Harrison's Principles of Internal Medicine, 16th Edition, The McGraw-Hill Companies.
25. Kronenberg et al., Williams Textbook of Endocrinology. Elsevier; 2008.
26. ARUP Laboratories (NATIONAL REFERENCE LABORATORY) www.aruplab.com.
27. Arnaldi et al., Diagnosis and complications of Cushing's syndrome: a consensus statement. J Clin Endocrinol Metab. 2003; 88:5593-5602.
28. Taylor et al., Validation of a High-Throughput Liquid Chromatography-Tandem Mass Spectrometry Method for Urine Cortisol and Cortisone. Clinical Chemistry 2002; 48:1511-1519.
29. Ghany M J, J. Liver Disease. McGraw-Hill; 2010.
30. Negoro et al., Urinary bile acid sulfate levels in patients with hepatitis C virus-related chronic liver diseases. Hepatol Res. 2009;39:760-765.
31. Simko et al., Random urine bile acids in prediction of liver abnormality in asymptomatic alcoholics. Arch Intern Med. 1988;148:312-315.
32. Kawai et al., Efficacy of urine bile acids as a non-invasive indicator of liver damage in rats. J. Tox. Sci. 2009, 34, 27-38 and references therein.
33. Online metabolic and molecular bases of inherited disease. Editors: Valle, Beaudet, Vogelstein, Kinzler, Antonarakis, Ballabio. Chapter 159: Congenital Adrenal Hyperplasia. Based on eight edition, volume II, pg. 1-67.
34. Online metabolic and molecular bases of inherited disease. Editors: Valle, Beaudet, Vogelstein, Kinzler, Antonarakis, Ballabio. Chapter 123: Inborn errors in bile Acid Biosynthesis and Storage of sterols other than cholesterol. Based on eight edition, volume II, pg. 2981-2961.
35. National Newborn Screening & Genetics Resource Center http: www. genes-r-us.uthscsa.edu.
36. Heubi et al., Inborn errors of bile acid metabolism. Semin Liver Dis. 2007;27:282-294.
37. Caulfield et al., 2002. The diagnosis of congenital adrenal hyperplasia in the newborn by gas chromatography/mass spectrometry analysis of random urine specimens Journal of Clinical Endocrinology & Metabolism 87:3682-3690.
38. Li et al., 2008 “Selecting Aptamers for a Glycoprotein through Incorporation of the Boronic Acid Moiety” J. Am. Chem. Soc. 130: 12636-12638.
39. Shangguan et al., 2008 “Cell-specific aptamer probes for membrane protein elucidation in cancer cells” J. Proteome Res. 7: 2133-2139.
40. Schauer et al., 2001. A cross-reactive, class-selective enzymatic array assay. Journal of the American Chemical Society 123:9443-9444.
41. Zhang et al., “A Colorimetric Sensor Array for Organics in Water”, J. Am. Chem. Soc. 2005, 127, 11548-11549.
42. Rakow et al., “Molecular Recognition and Discrimination of Amines with a Colorimetric Array” Angew. Chem. Int. Ed. 2005, 44, 4528-4532.
43. Folmer-Andersen et al., Pattern-based discrimination of enantiomeric and structurally similar amino acids: an optical mimic of the mammalian taste response J. Am. Chem. Soc. 2006, 128, 5652.
44. Baldini et al., 2004. Pattern-based detection of different proteins using an array of fluorescent protein surface receptors. Journal of the American Chemical Society 126:5656-5657.
45. Buryak et al., A chemosensor array for the colorimetric identification of 20 natural amino acids. J. Am. Chem. Soc. 2005, 127, 3700.
46. You et al., 2007. Detection and identification of proteins using nanoparticle-fluorescent polymer ‘chemical nose’ sensors. Nature Nanotechnology 2, 318-323.
47. Wright et al., 2005, Differential Receptors Create Patterns That Distinguish Various Proteins. Angew. Chem. Int. Ed. 44, 6375-6378.
48. Lee et al., 2006 Colorimetric Identification of Carbohydrates by a pH Indicator/pH Change Inducer Ensemble. Angew. Chem. Int. Ed., 45, 6485-6487.
49 Schiller et al., 2007, A Fluorescent Sensor Array for Saccharides Based on Boronic Acid Appended Bipyridinium Salts, Angew. Chjem. Int. Ed. 46, 1-4.
50. Edwards et al., 2007, Boronic Acid based Peptidic Receptors for Pattern-Based Saccharide Sensing in Neutral Aqueous Medica, an Application in real-Life Samples. J. Am. Chem. Soc. 129, 12575-12583.
51. Lavigne et al., 1998.—Based Analysis of Multiple Analytes by a Sensor Array: Toward the Development of an ‘Electronic Tongue’, 1998. J. Am. Chem. Soc. 120, 6429-6430.
52. Goodie et al., 2001. Development of Multianalyte Sensor Arrays of Chemically Derivatized Polymeric Microspheres Localized in Micromachined Cavities J. Am. Chem. Soc. 23, 2559-2570.
53. Du et al., 2004. Preparation of steroid antibodies and parallel detection of multianabolic steroid abuse with conjugated hapten microarray. 6166-6171.
54. Rochat et al., Cross-Reactive Sensor Arrays for the Detection of Peptides in Aqueous Soluiton by Fluorescence Spectroscopy. Chem. Eur. J. 2010, 16: 104-113.
55. Nutiu et al., 2003. Structure-switching signaling aptamers. J Am Chem Soc 125:4771-4778.
56. Nutiu et al., In vitro selection of structure-switching signaling aptamers. 2005. Angew. Chem. Int. Edu 1061-1065.
57. Liu et al., Fast Colorimetric Sensing of Adenosine and Cocaine Based on a General Sensor Design Involving Aptamers and Nanoparticles. Angew. Chem. 2006, 45, 90-94.
58. Wernette et al., Functional-DNA-Based Nanoscale Materials and Devices for Sensing Trace Contaminants in Water. MRS Bulletin, January 2008, 34-41.
59. MacMillan et al., 1990 “Synthesis of Functionally Tethered Oligonucleotides by the Convertible Nucleoside Approach” J. Org. Chem., 55, 5931-5933.
60. Jayasena, S. D. 1999. “Aptamers: an emerging class of molecules that rival antibodies in diagnostics [review]: Clin Chem 45:1628-1650.
61. Tan et al., 2000. “Molecular beacons: a novel DNA probe for nucleic acid and protein studies” 1107-1111.
62. Tyagi et al., “Molecular beacons: Probes that fluoresce upon hybridization.” Nature Biotechnoogy 1996, 14, 303-309.
63. Duda et al., Pattern Classification 2001.
64. Huber, P. J.; The Annals of Statistics 1985. 13(2):435-475.
65. Knight et al., Array-based evolution of DNA aptamers allows modeling of an explicit sequence fitness landscape. Nucleic Acids. Res. 2009, 37: e6.
66. Simko et al., Urinary bile acids in population screening for inapparent liver disease. Hepatogastroenterology. 1998;45:1706-1714.
67. Heubi et al., Tauroursodeoxycholic acid (TUDCA) in the prevention of total parenteral nutrition-associated liver disease. J. Pediatr. 2002;141:237-242.
68. Kowdley KV. Ursodeoxycholic acid therapy in hepatobiliary disease. Am J. Med. 2000; 108:481-486.
69. Cowles et al., Reversal of intestinal failure-associated liver disease in infants and children on parenteral nutrition: experience with 93 patients at a referral center for intestinal rehabilitation. J Pediatr Surg; 45:84-87; discussion 87-88.

What is claimed is:

1. A cross-reactive sensor array composition comprising:
(A) a first aptamer-based receptor capable of binding a first analyte; and
(B) a second aptamer-based receptor capable of binding a second analyte;
wherein binding of the first aptamer-based receptor to the first analyte is indicative of a disease state and binding of the second aptamer-based receptor to the second analyte is indicative of the absence of the disease state.

2. A method of detection a disease state comprising contacting a sample to a cross-reactive sensor array composition comprising:
(A) a first aptamer-based receptor capable of binding a first analyte if the first analyte is present in the sample; and
(B) a second aptamer-based receptor capable of binding a second analyte if the second analyte is present in the sample;
wherein binding of the first aptamer-based receptor to the first analyte is indicative of a disease state and binding of the second aptamer-based receptor to the second analyte is indicative of the absence of the disease state

3. A method of detection a disease state comprising contacting a sample to a cross-reactive sensor array composition comprising:
(A) a first collection of aptamer-based receptors capable of binding a first collection of analytes, if present, in the sample; and
(B) a second collection of aptamer-based receptors capable of binding a second collection of analytes, if present, in the sample;
wherein the binding pattern of individual receptors present in the first collection aptamer-based receptors of analytes of the first collection of analytes present in the sample are indicative of a disease state and wherein the binding pattern of individual receptors present in the second collection aptamer-based receptors of analytes of the second collection of analytes present in the sample are indicative of the absence of the disease state.


SEQUENCE LISTING (19 sequences)

SEQ ID NO: 1 - DNA, 78 bp, Artificial Sequence
  Description of Artificial Sequence Synthetic oligonucleotide
ggtattgaggaggctctcgggacgacnnggattttccnnnnacgaagtnngtcgtcccgatcctcctaacgtacgact

SEQ ID NO: 2 - DNA, 24 bp, Artificial Sequence
  Description of Artificial Sequence Synthetic primer
ggtattgaggaggctctcgggacg

SEQ ID NO: 3 - DNA, 20 bp, Artificial Sequence
  Description of Artificial Sequence Synthetic primer
agtcgtacgttaggaggatc

SEQ ID NO: 4 - DNA, 20 bp, Artificial Sequence
  Description of Artificial Sequence Synthetic primer
agtcgtacgttaggaggatc

SEQ ID NO: 5 - DNA, 25 bp, Artificial Sequence
  Description of Artificial Sequence Synthetic oligonucleotide
ggtccgtcgtcccgagagccggacc

SEQ ID NO: 6 - DNA, 28 bp, Artificial Sequence
  Description of Artificial Sequence Synthetic oligonucleotide
gtcaggattttcctccacgaagtgggac

SEQ ID NO: 7 - DNA, 28 bp, Artificial Sequence
  Description of Artificial Sequence Synthetic oligonucleotide
gtcnngattttcnnnnacgaagtnngac

SEQ ID NO: 8 - DNA, 15 bp, Artificial Sequence
  Description of Artificial Sequence Synthetic oligonucleotide
gtcgtcccgagagcc

SEQ ID NO: 9 - DNA, 47 bp, Artificial Sequence
  Description of Artificial Sequence Synthetic oligonucleotide
ggctctcgggacgacnnggattttccnnnncagaatgnngtcgtccc

SEQ ID NO: 10 - DNA, 13 bp, Artificial Sequence
  Description of Artificial Sequence Synthetic oligonucleotide
cgtcccgagagag

SEQ ID NO: 11 - DNA, 44 bp, Artificial Sequence
  Description of Artificial Sequence Synthetic oligonucleotide
ctctctcgggacgctgggttttcccaggacgaagtccgtcccga

SEQ ID NO: 12 - DNA, 13 bp, Artificial Sequence
  Description of Artificial Sequence Synthetic oligonucleotide
gtcccgagagcag

SEQ ID NO: 13 - DNA, 44 bp, Artificial Sequence
  Description of Artificial Sequence Synthetic oligonucleotide
ctgctctcgggacgtggattttccgcatacgaagttgtcccgag

SEQ ID NO: 14 - DNA, 47 bp, Artificial Sequence
  Description of Artificial Sequence Synthetic oligonucleotide
ctgctctcgggacgtggattttccacaaaccagaatggtgtcccgag

SEQ ID NO: 15 - DNA, 13 bp, Artificial Sequence
  Description of Artificial Sequence Synthetic oligonucleotide
gtcgtcccgagag

SEQ ID NO: 16 - DNA, 47 bp, Artificial Sequence
  Description of Artificial Sequence Synthetic oligonucleotide
ctctcgggacgacatggattttccatcaacgaagtgcgtccgtcccg

SEQ ID NO: 17 - DNA, 45 bp, Artificial Sequence
  Description of Artificial Sequence Synthetic oligonucleotide
ccctgctgggtgaagtaactccttttaggacagcagggctctcgg

SEQ ID NO: 18 - DNA, 25 bp, Artificial Sequence
  Description of Artificial Sequence Synthetic oligonucleotide
ccagtccgagagccctgctgactgg

SEQ ID NO: 19 - DNA, 45 bp, Artificial Sequence
  Description of Artificial Sequence Synthetic oligonucleotide
ggctctcgggacgacnngattttcnnnnatgaagtnngtcgtccc